(19) United States
(12) Patent Application Publication
(10) Pub. No.: US 2009/0062546 A1
(43) Pub. Date: Mar. 5, 2009

(54) Dolasetron trifluoroacetate, polymorphs of dolasetron trifluoroacetate and process for preparation thereof

(76) Inventors: Janos Hajko, Debrecen (HU); Tivadar Tamas, Debrecen (HU); Adrienne Kovacsne-Mezei, Debrecen (HU); Piroska Kovacs, Debrecen (HU)

Correspondence Address: KENYON & KENYON LLP, ONE BROADWAY, NEW YORK, NY, 10004, US

(21) Appl. No.: 12/220,104

(22) Filed: Jul. 21, 2008

Related U.S. Application Data
(60) Provisional application No. 60/961,383, filed on Jul. 20, 2007.
(60) Provisional application No. 60/980,661, filed on Oct. 17, 2007.
(60) Provisional application No. 60/986,485, filed on Nov. 8, 2007.
(60) Provisional application No. 61/002,407, filed on Nov. 7, 2007.
(60) Provisional application No. 61/014,160, filed on Dec. 17, 2007.
(60) Provisional application No. 61/014,178, filed on Dec. 17, 2007.
(60) Provisional application No. 61/018,612, filed on Jan. 2, 2008.
(60) Provisional application No. 61/021,758, filed on Jan. 17, 2008.
(60) Provisional application No. 61/043,878, filed on Apr. 10, 2008.

(51) Int. Cl. C07D 221/22

(52) U.S. Cl. 546/97

(57) ABSTRACT

Provided are crystalline forms of dolasetron trifluoroacetate, methods for their preparation, and their use in preparing dolasetron mesylate.

### CROSS REFERENCE TO RELATED APPLICATIONS

The present invention claims the benefit of the following U.S. Provisional Patent Application Nos.: 60/961,383, filed Jul. 20, 2007; 60/980,661, filed Oct. 17, 2007; 60/986,485, filed Nov. 8, 2007; 61/014,160, filed Dec. 17, 2007; 61/043,878, filed Apr. 10, 2008; 61/002,407, filed Nov. 7, 2007; 61/014,178, filed Dec. 17, 2007; 61/018,612, filed Jan. 2, 2008; and 61/021,758, filed Jan. 17, 2008. The contents of these applications are incorporated herein by reference.

### FIELD OF THE INVENTION

The present invention relates to dolasetron trifluoroacetate and polymorphic forms of dolasetron trifluoroacetate, and processes for preparing said forms.

### BACKGROUND OF THE INVENTION

Dolasetron mesylate monohydrate, (2α,6α,8α,9αβ)-octahydro-3-oxo-2,6-methano-2H-quinolizin-8-yl-1H-indole-3-carboxylate monomethanesulfonate monohydrate (referred to as DLS-MsOH.H₂O) a compound having the following chemical structure,

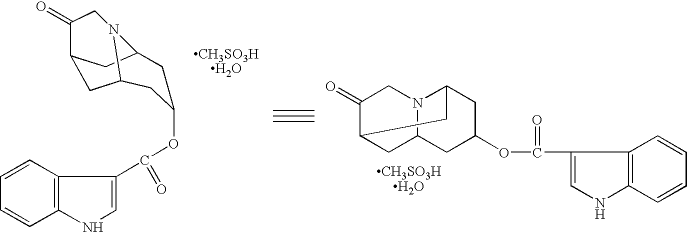

is a serotonin receptor (5-HT₃) antagonist used as an antiemetic and antinauseant agent in chemo- and radiotherapies.

Dolasetron mesylate (DLS-MsOH) developed by Merrell Dow Pharmaceuticals is marketed as tablets for oral administration and as sterile solution for intravenous administration by Aventis, under the trade name Anzemet®.

EP patents no. 0339669 and 0266730 report the preparation and crystallization of dolasetron mesylate. Polymorphs of dolasetron mesylate are described in the publications WO2006/026927, WO2007/072506, and WO2007/081909. Polymorphs of dolasetron base are described in WO2007/072507 and WO 2007/081907.

The present invention relates to polymorphs of dolasetron trifluoroacetate, a new intermediate for preparing dolasetron mesylate.

The occurrence of different crystal forms (polymorphism) is a property of some molecules and molecular complexes. A single molecule may give rise to a variety of solids having distinct physical properties like melting point, X-ray diffraction pattern and infrared absorption fingerprint. The differences in the physical properties of polymorphs result from the orientation and intermolecular interactions of adjacent molecules (complexes) in the bulk solid.

Accordingly, polymorphs are distinct solids sharing the same molecular formula yet having distinct advantageous and/or disadvantageous physical properties compared to other forms in the polymorph family. One of the most important physical properties of pharmaceutical polymorphs is their solubility in aqueous solution, which may influence the bioavailability of the drug.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

The discovery of new polymorphic forms of dolasetron trifluoroacetate provides a new opportunity to improve the performance of the synthesis of the active pharmaceutical ingredient (API), dolasetron mesylate, by producing polymorphs of dolasetron trifluoroacetate having improved characteristics, such as flowability, and solubility. Thus, there is a need in the art for polymorphic forms of dolasetron trifluoroacetate.

### SUMMARY OF THE INVENTION

In one embodiment, the present invention encompasses dolasetron trifluoroacetate

In another embodiment, the present invention encompasses an isolated dolasetron trifluoroacetate.

In another embodiment, the invention encompasses the preparation of dolasetron mesylate from dolasetron trifluoroacetate.

In one embodiment, the present invention encompasses crystalline dolasetron trifluoroacetate characterized by data selected from the group consisting of: a powder XRD pattern with peaks at about 16.4, 16.6, 19.1, and 21.6±0.2 degrees 2-theta, a powder XRD pattern as depicted in FIG. 1; and a combination thereof.

In another embodiment, the present invention provides a process for preparing crystalline dolasetron trifluoroacetate characterized by data selected from the group consisting of: a powder XRD pattern with peaks at about 16.4, 16.6, 19.1, and 21.6±0.2 degrees 2-theta, a powder XRD pattern as depicted in FIG. 1; and a combination thereof comprising crystallizing dolasetron trifluoroacetate from a mixture comprising Indole-3-carboxylic acid having the following structure;

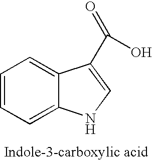

toluene, trifluoroacetic acid, trifluoroacetic anhydride and endo-5-hydroxy-8-azatricyclo[5.3.1.0^{3,8}]-undecan-10-one having the following structure;

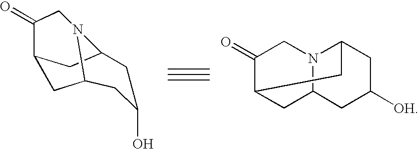

In one embodiment, the present invention provides crystalline dolasetron trifluoroacetate characterized by data selected from the group consisting of: a powder XRD pattern with peaks at about 12.0, 15.3, 18.1, and 25.6±0.2 degrees 2-theta, a powder XRD pattern as depicted in FIG. 3; and a combination thereof.

In another embodiment, the present invention provides a process for preparing crystalline dolasetron trifluoroacetate characterized by data selected from the group consisting of: a powder XRD pattern with peaks at about 12.0, 15.3, 18.1, and 25.6±0.2 degrees 2-theta, a powder XRD pattern as depicted in FIG. 4; and a combination thereof comprising drying crystalline dolasetron trifluoroacetate characterized by data selected from the group consisting of: a powder XRD pattern with peaks at about 9.0, 18.1, 18.5, 19.4, and 20.4±0.2 degrees 2-theta, a powder XRD pattern as depicted in FIG. 12, and combination thereof.

In yet another embodiment, the present invention provides a process for preparing crystalline dolasetron trifluoroacetate characterized by data selected from the group consisting of: a powder XRD pattern with peaks at about 12.0, 15.3, 18.1, and 25.6±0.2 degrees 2-theta, a powder XRD pattern as depicted in FIG. 4; and a combination thereof comprising crystallizing dolasetron trifluoroacetate from ethanol and drying the obtained dolasetron trifluoracetate to obtain said crystalline dolasetron trifluoroacetate.

In one embodiment, the present invention provides crystalline dolasetron trifluoroacetate characterized by data selected from the group consisting of: a powder XRD pattern with peaks at about 5.0, 11.4, 17.2, and 18.5±0.2 degrees 2-theta, a powder XRD pattern as depicted in FIG. 8; and a combination thereof.

In another embodiment, the present invention provides a process for preparing the crystalline dolasetron trifluoroacetate characterized by data selected from the group consisting of: a powder XRD pattern with peaks at about 5.0, 11.4, 17.2, and 18.5±0.2 degrees 2-theta, a powder XRD pattern as depicted in FIG. 8; and a combination thereof, comprising crystallizing dolasetron trifluoroacetate from isopropanol.

In one embodiment, the present invention encompasses crystalline dolasetron trifluoroacetate characterized by data selected from the group consisting of: a powder XRD pattern with peaks at about 9.0, 18.1, 18.5, 19.4, and 20.4±0.2 degrees 2-theta, and a powder XRD pattern as depicted in FIG. 12; and a combination thereof.

In another embodiment, the present invention provides a process for preparing crystalline dolasetron trifluoroacetate characterized by data selected from the group consisting of: a powder XRD pattern with peaks at about 9.0, 18.1, 18.5, 19.4, and 20.4±0.2 degrees 2-theta, and a powder XRD pattern as depicted in FIG. 12; and a combination thereof comprising crystallizing dolasetron trifluoroacetate from ethanol.

In another embodiment, the present invention encompasses a process for preparing dolasetron mesylate from any one of the above forms of dolasetron trifluoroacetate or mixtures thereof.

In yet another embodiment, the present invention provides a process for preparing dolasetron mesylate by preparing any one of the above forms of dolasetron trifluoroacetate and mixtures thereof according to the process of the present invention, and converting them to dolasetron mesylate.

### BRIEF DESCRIPTION OF THE FIGURES

FIG. 1 shows a powder XRD pattern of crystalline dolasetron trifluoroacetate designated Form 1.

FIG. 2 shows a FT-IR spectrum of dolasetron trifluoroacetate designated Form 1.

FIG. 3 shows a powder XRD pattern of different batches of crystalline dolasetron trifluoroacetate designated Form 1.

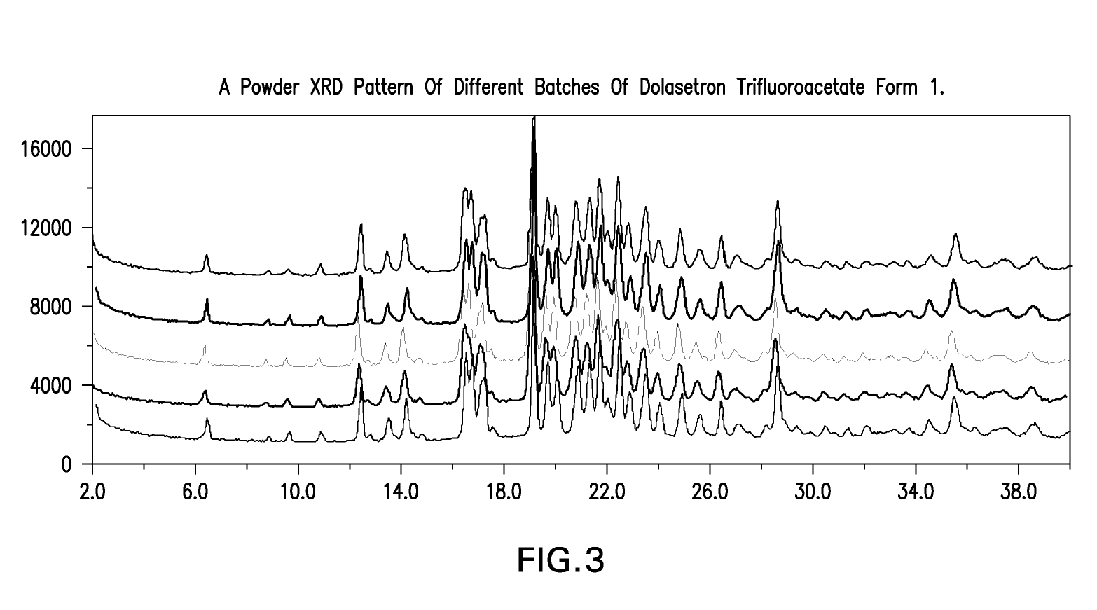

FIG. 4 shows a powder XRD pattern of crystalline dolasetron trifluoroacetate designated Form 2.

FIG. 5 shows a FT-IR spectrum of crystalline dolasetron trifluoroacetate designated Form 2.

FIG. 6 shows a DSC calorimetrogram of dolasetron trifluoroacetate designated Form 2.

FIG. 7 shows a TGA thermogram of crystalline dolasetron trifluoroacetate designated Form 2.

FIG. 8 shows a powder XRD pattern of crystalline dolasetron trifluoroacetate designated Form 3.

FIG. 9 shows FT-IR spectrum of crystalline dolasetron trifluoroacetate designated Form 3.

FIG. 10 shows a DSC calorimetrogram of crystalline dolasetron trifluoroacetate designated Form 3.

FIG. 11 shows a TGA thermogram of crystalline dolasetron trifluoracetate designated Form 3.

FIG. 12 shows a powder XRD pattern of crystalline dolasetron trifluoroacetate designated Form 4.

FIG. 13 shows a FT-IR spectrum of crystalline dolasetron trifluoroacetate designated Form 4

FIG. 14 shows a powder XRD pattern of crystalline dolasetron base designated Form G.

### DETAILED DESCRIPTION OF THE INVENTION

The present invention relates to dolasetron trifluoroacetate, polymorphic forms of dolasetron trifluoroacetate, and processes for preparing said forms.

In one embodiment, the present invention encompasses dolasetron trifluoroacetate.

Preferably, dolasetron trifluoroacetate is provided in an isolated form. Preferably, the isolated dolasetron trifluoroacetate is solid, more preferably, it is crystalline, providing a composition of dolasetron trifluoroacetate containing less than about 1% by weight, more preferably, less than about 0.5% by weight, most preferably, less than about 0.1% by weight of starting materials or dolasetron base. As used herein, the term “isolated” in reference to dolasetron trifluoroacetate corresponds to dolasetron trifluoroacetate that is physically separated from the reaction mixture in which it is formed. For example, the separation can be done by filtering the precipitated dolasetron trifluoroacetate. More preferably the dolasetron trifluoroacetate is separated from dolasetron base.

In a preferred embodiment, the present invention encompasses solid dolasetron trifluoroacetate.

In a preferred embodiment, the present invention encompasses crystalline dolasetron trifluoroacetate.

Preferably, dolasetron trifluoroacetate is characterized by data selected from the group consisting of a ¹H-NMR (500.13 MHz) spectrum in (CD₃)₂SO having chemical shifts at about δ 2.27-2.63 (m, 9H); 2.78 (s, 1H), 4.00 (m, 2H); 4.10 (s, 2H); 5.40 (s, 1H); 7.22 (m, 2H), 7.55 (m, 1H); 8.08 (m, 2H); 12.14 (s, 1H), a ¹³C-NMR (125.78 MHz) spectrum in (CD₃)₂SO having chemical shifts at about δ 25.37, 31.72, 37.54, 49.45, 58.96, 62.80, 105.84, 112.48, 120.02, 121.40, 122.52, 125.92, 132.45, 136.49, 158.86, 159.21, 162.93, 204.89; or a combination thereof.

The above dolasetron trifluoroacetate can be used to prepare dolasetron mesylate as will be illustrated below.

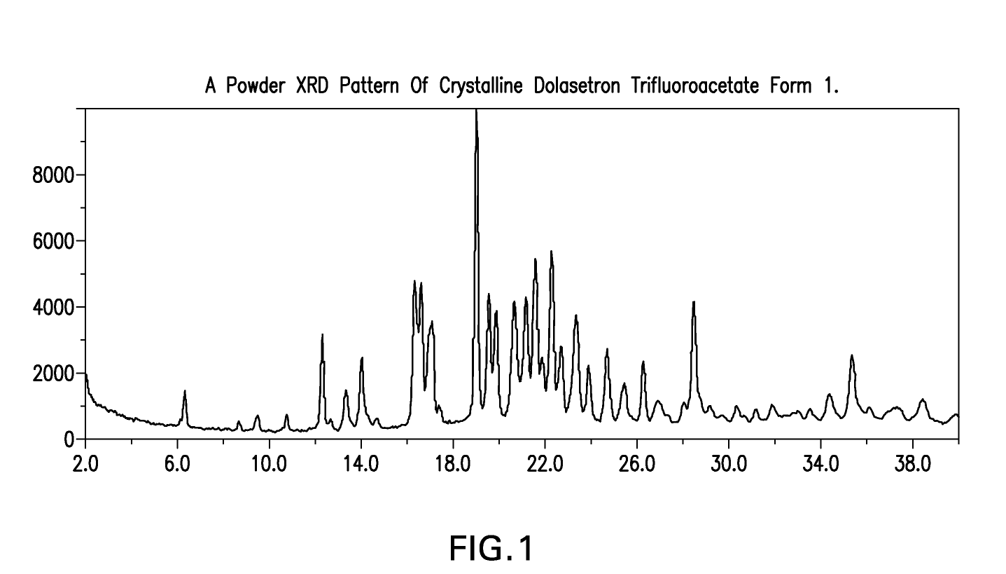

The present invention encompasses crystalline dolasetron trifluoroacetate, designated Form 1, characterized by data selected from the group consisting of: a powder XRD pattern with peaks at about 16.4, 16.6, 19.1, and 21.6±0.2 degrees 2-theta, a powder XRD pattern as depicted in FIG. 1; and a combination thereof.

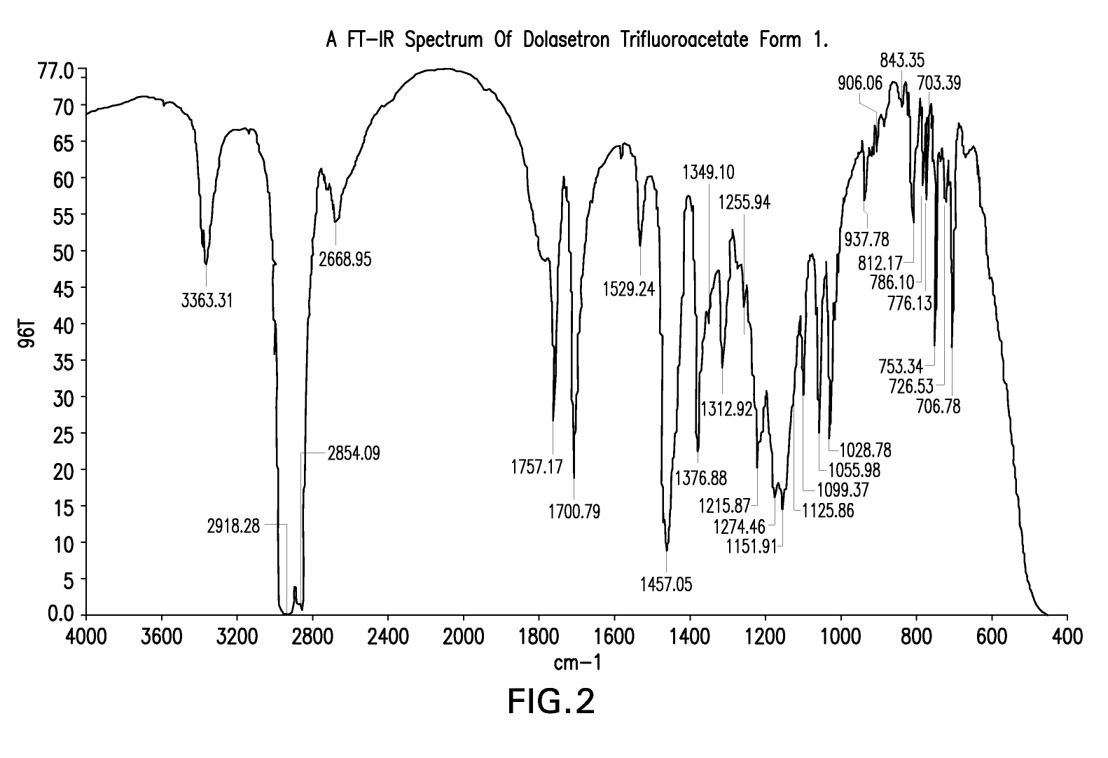

The above crystalline Form 1 of dolasetron trifluoroacetate can be further characterized by data selected from the group consisting of: a powder XRD pattern with peaks at about 12.4, 19.6, 20.0, 20.8, 22.4, and 28.6±0.2 degrees 2-theta, a FT-IR spectrum with peaks at about 3363, 1757, 1704, 1529, 1312, 1216, 1175, 1152, 1056 and 1029 cm⁻¹±2 cm⁻¹, a FT-IR spectrum as depicted in FIG. 2; and a combination thereof.

Preferably, dolasetron trifluoroacetate Form 1 may be further characterized as anhydrous containing less than about 1% of water, more preferably, less than 0.7% of water by weight, as measured by TGA.

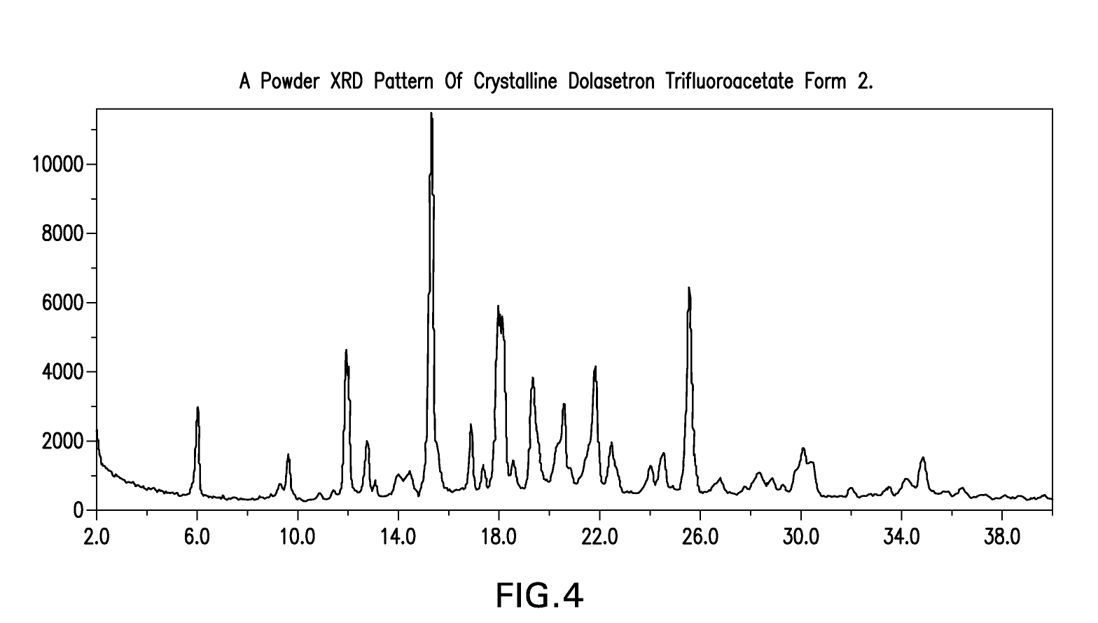

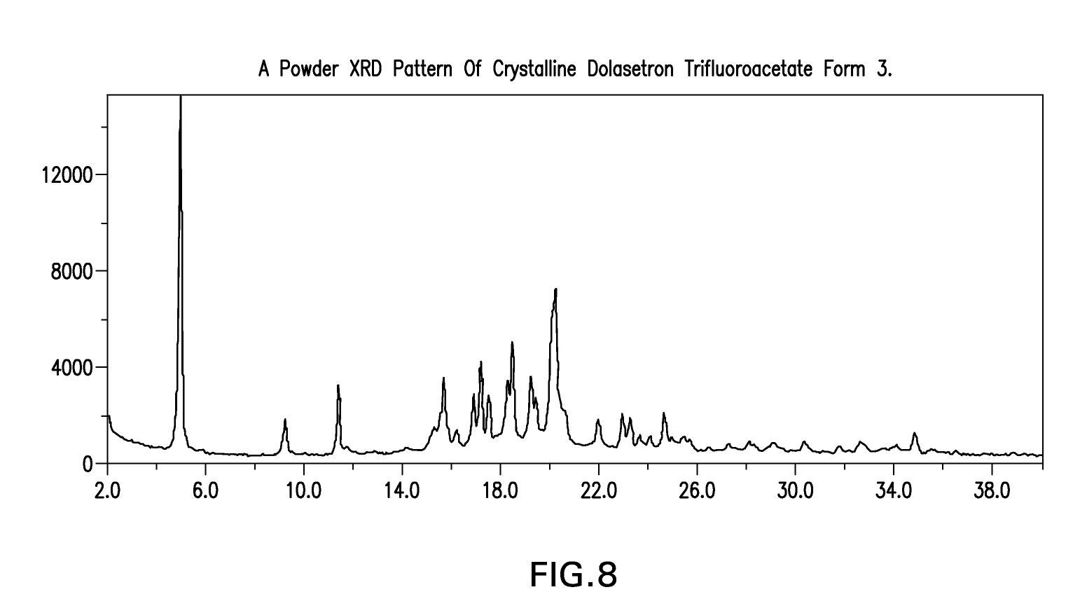

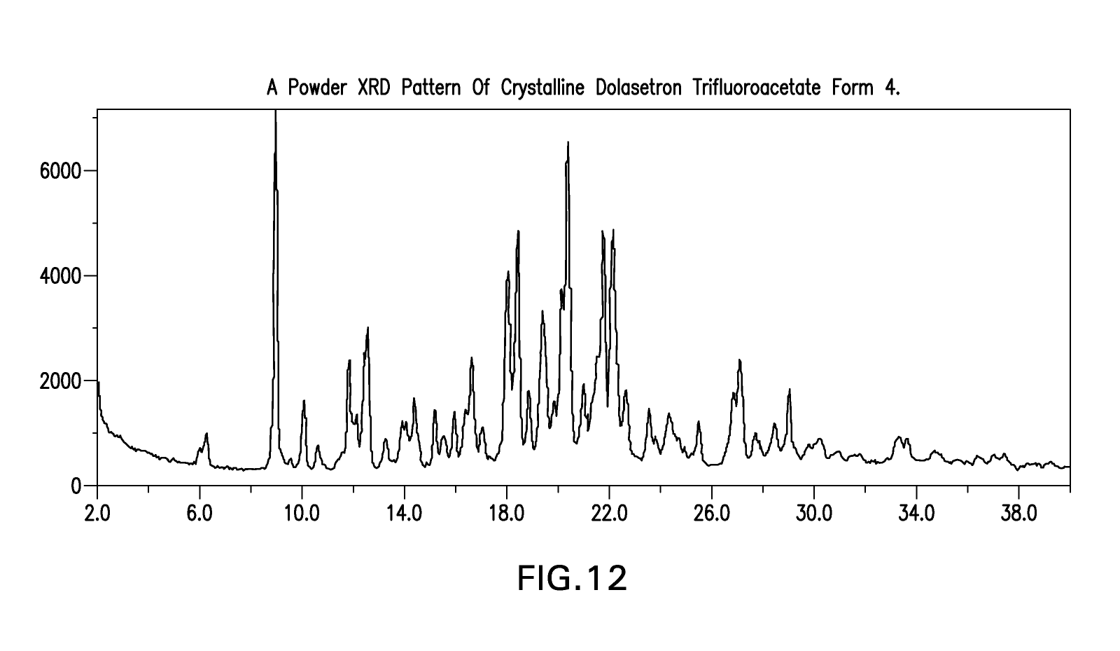

The above crystalline dolasetron trifluoroacetate Form 1 has less than about 10% by weight, more preferably less than about 5% by weight, and most preferably less than about 1% by weight of crystalline dolasetron trifluoroacetate selected from the group consisting of: crystalline dolasetron trifluoroacetate, designated Form 2, characterized by data selected from the group consisting of: a powder XRD pattern with peaks at about 12.0, 15.3, 18.1, and 25.6±0.2 degrees 2-theta, a powder XRD pattern as depicted in FIG. 4, and a combination thereof; a crystalline dolasetron trifluoroacetate, designated Form 3, characterized by data selected from the group consisting of: a powder XRD pattern with peaks at about 5.0, 11.4, 17.2, and 18.5±0.2 degrees 2-theta, a powder XRD pattern as depicted in FIG. 8, and a combination thereof; and a crystalline dolasetron trifluoroacetate, designated Form 4, characterized by data selected from the group consisting of: a powder XRD pattern with peaks at about 9.0, 18.1, 18.5, 19.4, and 20.4±0.2 degrees 2-theta, a powder XRD pattern as depicted in FIG. 12, and a combination thereof; or mixtures thereof.

The content of crystalline dolatestron trifluoroacetate Form 2 in crystalline Form 1 is measured by PXRD using the peak at about 15.3±0.2 degrees two-theta; the content of crystalline Form 3 in crystalline Form 1 is measured by PXRD using the peak at about 5.0±0.2 degrees two-theta; and the content of crystalline Form 4 in crystalline Form 1 is measured by PXRD using the peak at about 9.0±0.2 degrees two-theta.

The above crystalline Form 1 of dolasetron trifluoroacetate can be prepared by a process comprising crystallizing dolasetron trifluoroacetate from a mixture comprising Indole-3-carboxylic acid of the following formula,

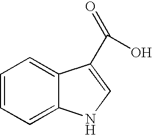

toluene, trifluoroacetic acid, trifluoroacetic anhydride and endo-5-hydroxy-8-azatricyclo[5.3.1.0^{3,8}]-undecan-10-one of the following formula

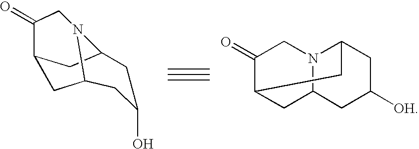

The above process of preparing crystalline dolasetron trifluoroacetate preferably comprises: a) adding Indole-3-carboxylic acid to a solution of trifluoroacetic anhydride in toluene and trifluoroacetic acid to form a mixture, b) combining the mixture with endo-5-hydroxy-8-azatricyclo[5.3.1.0^{3,8}]-undecan-10-one to obtain a solution, and c) precipitating the crystalline dolasetron trifluoroacetate.

In step a) Indole-3-carboxylic acid is added to a solution of trifluoroacetic anhydride in a mixture of toluene and trifluoroacetic acid. Preferably, the molar ratio in the mixture is about 1 to about 2 molar equivalents of trifluoroacetic anhydride and about 1 to about 1.5 molar equivalent of indole-3-carboxylic acid. Preferably, the addition is carried out at about 10° C. to about 40° C., more preferably at about 15° C. to about 30° C., most preferably at about 20° C. to about 25° C. Preferably, the addition is carried out in portions. Preferably, the addition is carried out over a period of about 5 minutes to about 60 minutes, more preferably of about 10 minutes to about 25 minutes, most preferably of about 10 minutes to about 20 minutes.

The mixture from step a) is combined with endo-5-hydroxy-8-azatricyclo[5.3.1.0^{3,8}]-undecan-10-one providing a solution. The solution is maintained at room temperature for a sufficient period of time to provide dolasetron trifluoroacetate.

As used herein, “room temperature” refers to a temperature of about 20° C. to about 25° C.

Preferably, the solution is maintained for a period of about 0.5 hour to about 20 hours, more preferably for about 1 hour to about 5 hours, most preferably for about 1.5 hours to about 3 hours. Preferably, the solution is maintained while stirring.

Precipatation in step c) may comprise removing the excess of trifluoroacetic acid, preferably, by evaporation under reduced pressure, to obtain the crystalline dolasetron trifluoroacetate, i.e providing a suspension comprising said crystalline Form 1 of dolasteron trifluoroacetate. Preferably, evaporation is carried out at a temperature of about 20° C. to about 80° C., more preferably, at a temperature of about 30° C. to about 70° C. Preferably, evaporation is carried out at a pressure of about 20 to about 500 mbar, more preferably of about 50 to about 200 mbar, most preferably of about 70 to about 120 mbar.

The process for preparing said crystalline dolasetron trifluoroacetate may further comprise recovering the crystalline dolasetron trifluoroacetate Form 1 from the obtained suspension. The recovery may be carried out for example, by filtering the suspension, washing, and drying. Preferably, drying is carried out at a temperature of about 40° C. to about 120° C., more preferably at about 50° C. to about 100° C., most preferably at about 75° C. to about 85° C. Preferably, drying is carried out for a period of about 1 hour to about 20 hours, more preferably of about 2 hours to about 10 hours, most preferably of about 3 hours to about 6 hours.

The present invention also encompasses crystalline dolasetron trifluoroacetate, designated Form 2, characterized by data selected from the group consisting of: a powder XRD pattern with peaks at about 12.0, 15.3, 18.1, and 25.6±0.2 degrees 2-theta, a powder XRD pattern as depicted in FIG. 4; and a combination thereof.

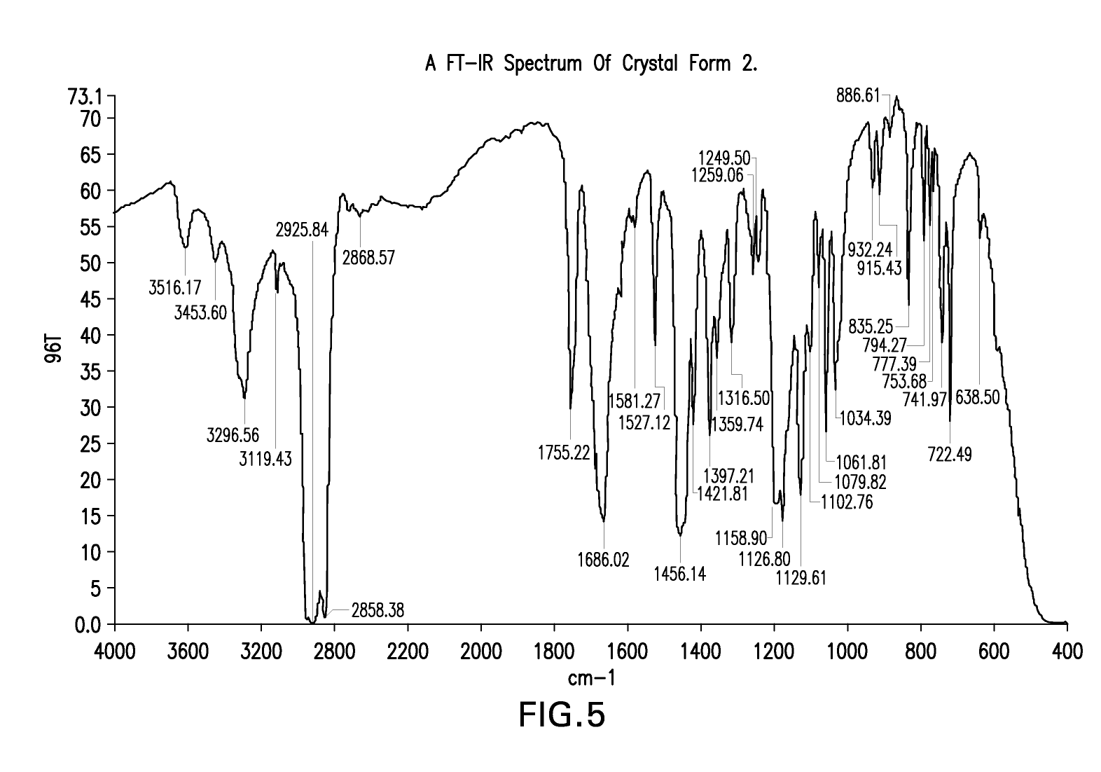

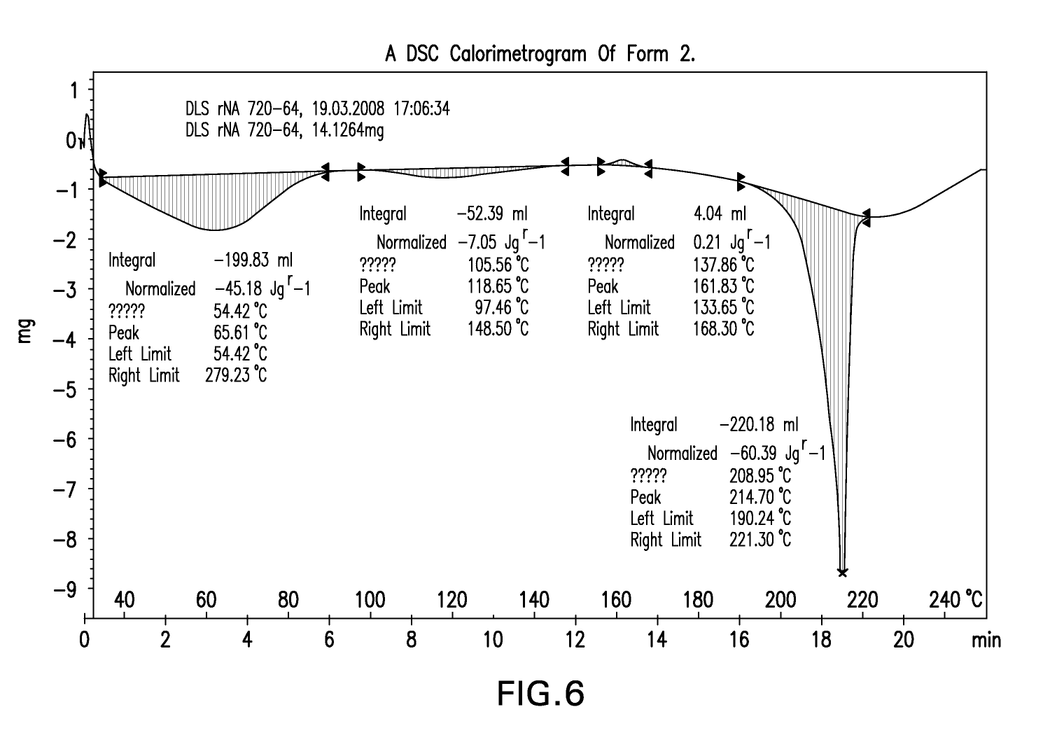

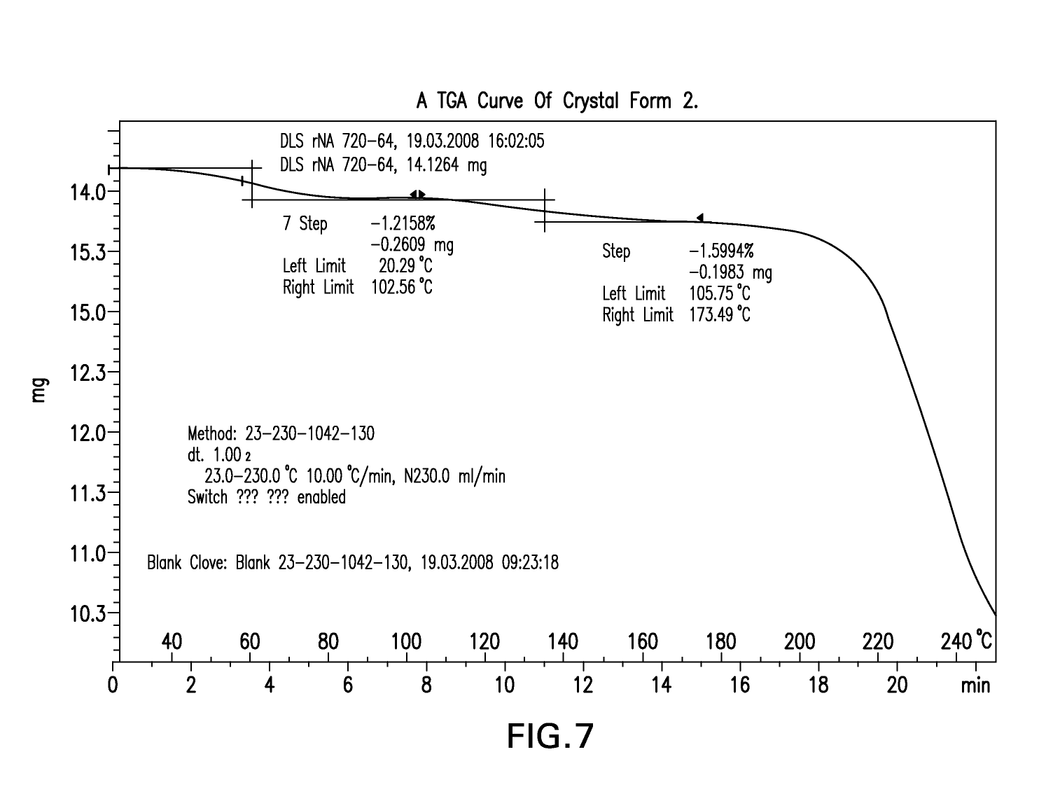

The above crystalline Form 2 of dolasetron trifluoroacetate can be further characterized by data selected from the group consisting of: a powder XRD pattern with peaks at about 6.0, 9.7, 16.9, 19.4, 20.6, and 21.8±0.2 degrees 2-theta; a FT-IR spectrum with peaks at about 3616, 3297, 1755, 1666, 1317, 1199, 1179, 1130, 1062 and 1035 cm⁻¹±2 cm⁻¹; a FT-IR spectrum as depicted in FIG. 5; a DSC calorimetrogram with two endothermic peaks at about 63° C. and about 119° C. due to desolvation, a small exothermic peak at about 162° C. and a sharp melting peak at about 215° C.; a DSC calorimetrogram as depicted in FIG. 6; a TGA thermogram as depicted in FIG. 7; and a combination thereof.

The above crystalline Form 2 of dolasetron trifluoroacetate may be further characterized as a solvate containing water and ethanol in non-stoichiometric amounts. Preferably, the content of Ethanol is about 0.2%, to about 1.4% by weight as determined by Gas Chromatography (“GC”) or TGA and the content of water is about 1.8% to about 2.8% by weight as measured by Karl Fischer or TGA.

The above crystalline dolasetron trifluoroacetate Form 2 has less than about 10% by weight, more preferably less than about 5% by weight, and most preferably less than about 1% by weight of crystalline dolasetron trifluoroacetate designated Forms 1, 3, 4 or mixtures thereof. The content of crystalline dolasetron trifluoroacetate Form 1 in crystalline Form 2 is measured by PXRD using the peak at about 19.1±0.2 degrees two-theta; the content of crystalline Form 3 in crystalline Form 2 is measured by PXRD using the peak at about 5.0±0.2 degrees two-theta; and the content of crystalline Form 4 in crystalline Form 2 is measured by PXRD using the peak at about 9.0±0.2 degrees two-theta.

The above crystalline dolasetron trifluoroacetate Form 2 can be prepared by a process comprising crystallizing dolasetron trifluoroacetate from ethanol and drying the obtained dolasetron trifluoracetate to obtain said crystalline dolasetron trifluoroacetate.

Preferably, the crystallization comprises providing a solution of dolasetron trifluoroacetate in ethanol, and precipitating the crystalline dolasetron trifluoroacetate to obtain a suspension.

Preferably, the solution of dolasetron trifluoroacetate in ethanol is provided by combining dolasetron trifluoroacetate and ethanol to obtain a mixture and heating the mixture. Preferably, heating is carried out to about reflux. Preferably, reflux is at a temperature of about 70° C. to about 90° C., more preferably at about 75° C. to about 82° C.

Preferably, precipitation is carried out by a process comprising cooling the solution to a temperature of about 20° C. to about −10° C., more preferably, to about 10° C. to about 0° C., most preferably, to about 8° C. to about 2° C. to obtain the suspension comprising of the crystalline dolasetron trifluoroacetate.

Preferably, the suspension is further maintained at the same temperature for about 1 hour to about 24 hours, more preferably, for about 6 hours to about 20 hours, most preferably, for about 8 hours to about 16 hours to increase the yield of the precipitated Form.

Preferably, drying is conducted at a temperature of about 40° C. to about 120° C., more preferably at about 50° C. to about 100° C., most preferably at about 75° C. to about 85° C. Preferably, drying is carried out for about 1 hour to about 24 hours, more preferably for about 6 hours to about 20 hours, most preferably for about 8 hours to about 16 hours. Drying is preferably carried out under reduced pressure, more preferably at a pressure of about 20 to about 500 mbar, even more preferably of about 20 to about 200 mbar, most preferably of about 20 to about 100 mbar. Prior to drying the obtained suspension comprising dolasetron trifluoroacetate may be filtered.

The above crystalline dolasetron trifluoroacetate Form 2 can also be prepared by a process comprising drying dolasetron trifluoroacetate Form 4 characterized by data selected from the group consisting of: a powder XRD pattern with peaks at about 9.0, 18.1, 18.5, 19.4, and 20.4±0.2 degrees 2-theta, a powder XRD pattern as depicted in FIG. 12; and a combination thereof. Preferably, drying is carried out at a temperature of about 40° C. to about 120° C., more preferably at about 50° C. to about 100° C., most preferably at about 75° C. to about 85° C.

Preferably, drying is carried out for about 1 hour to about 24 hours, more preferably for about 6 hours to about 20 hours, most preferably for about 8 hours to about 16 hours. Drying is preferably carried out under reduced pressure, more preferably at a pressure of about 20 to about 500 mbar, even more preferably of about 20 to about 200 mbar, most preferably of about 20 to about 100 mbar.

The present invention further encompasses crystalline dolasetron trifluoroacetate, designated Form 3, characterized by data selected from the group consisting of: a powder XRD pattern with peaks at about 5.0, 11.4, 17.2, and 18.5±0.2 degrees 2-theta, a powder XRD pattern as depicted in FIG. 8; and a combination thereof.

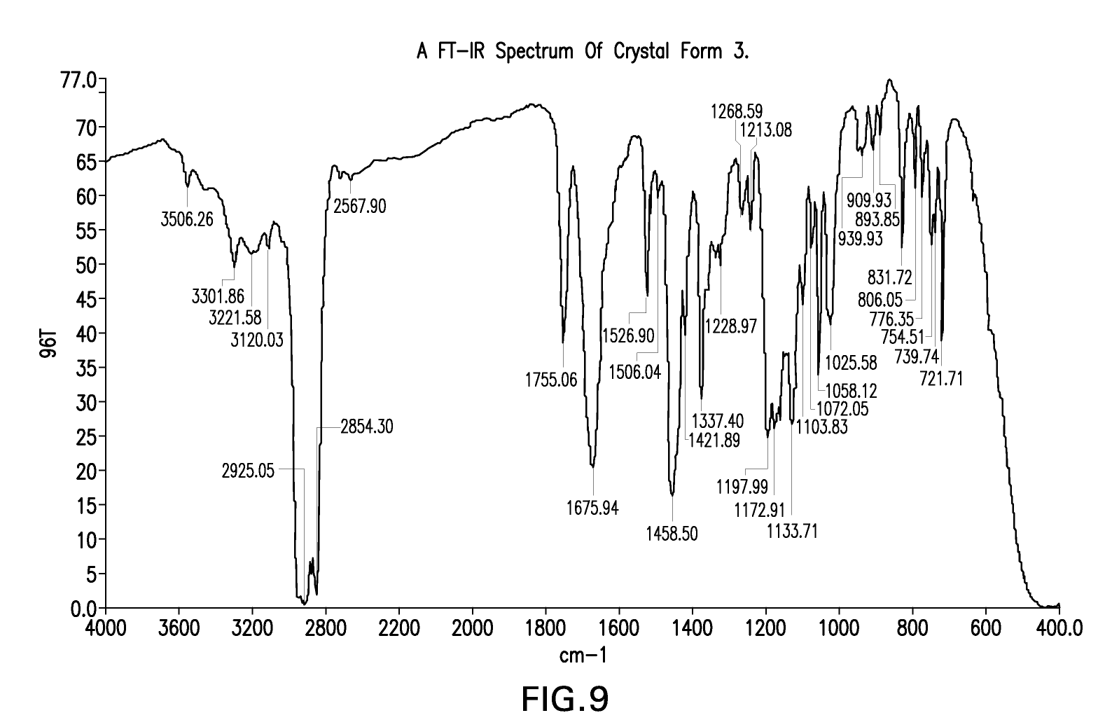

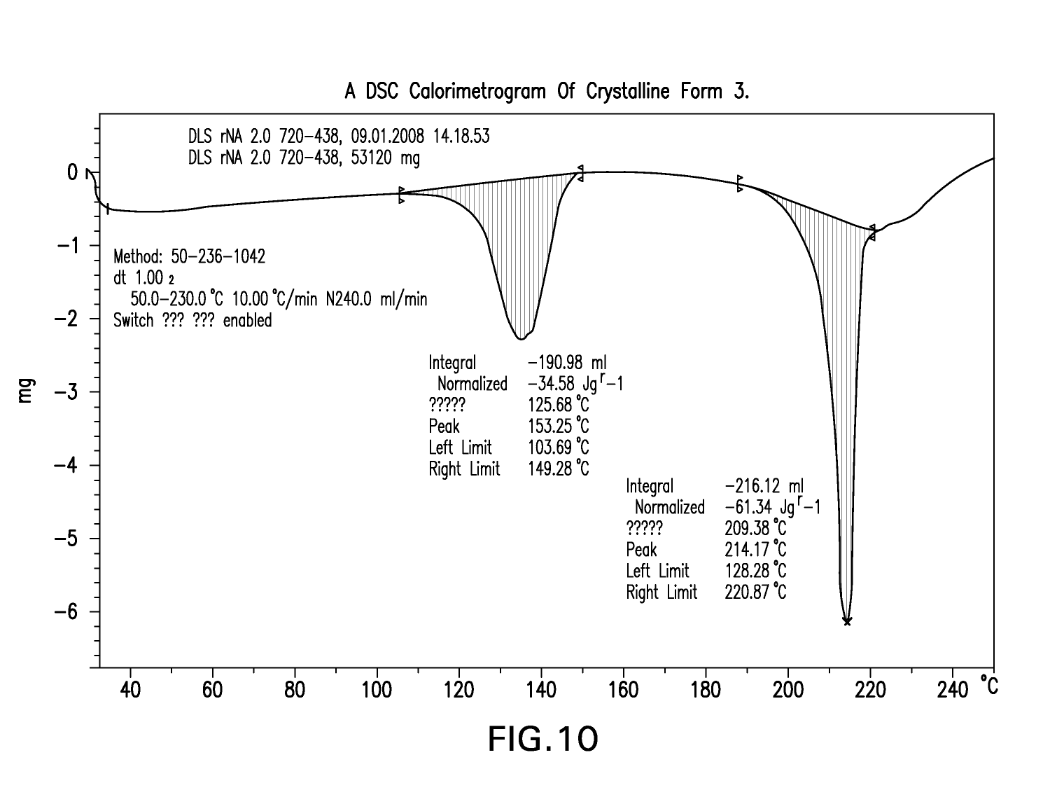

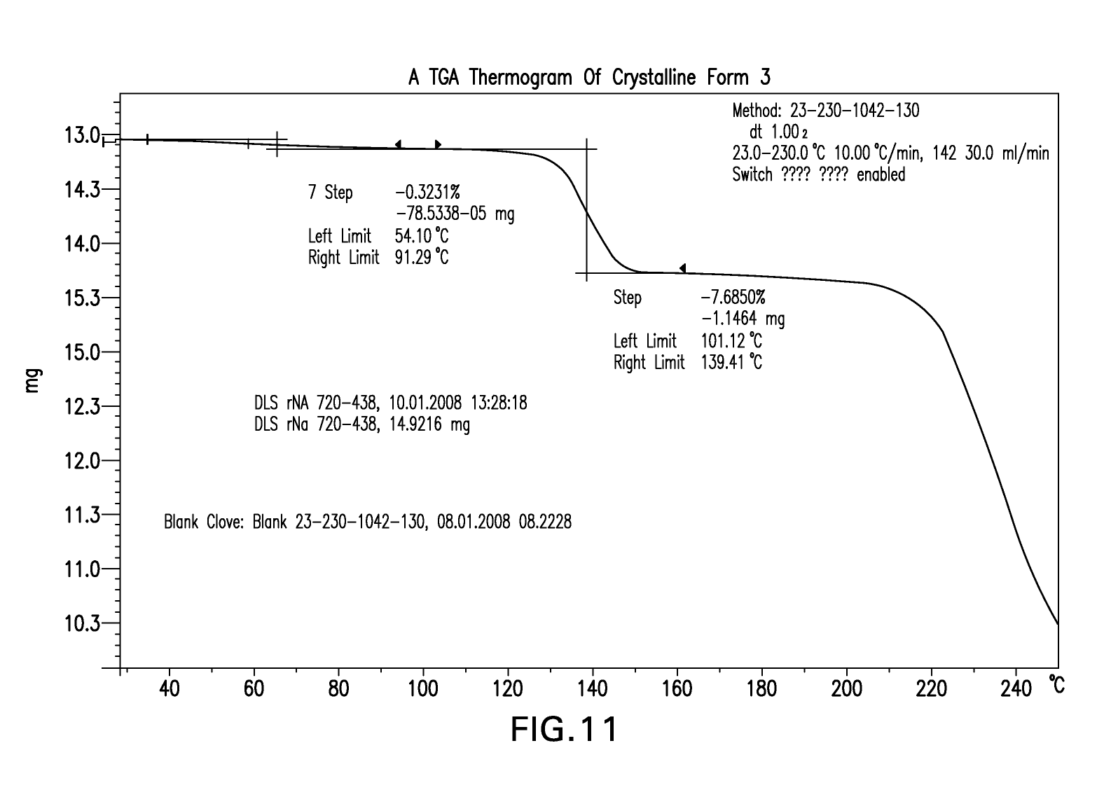

The above crystalline Form 3 of dolasetron trifluoroacetate can be further characterized by a powder XRD pattern with peaks at about 9.3, 15.7, 16.9, 17.5, 19.4, and 20.3±0.2 degrees 2-theta. a FT-IR spectrum with peaks at about 3560, 1755, 1676, 1527, 1269, 1198, 1178, 1131, 1058 and 1026 cm⁻¹±2 cm⁻¹; a FT-IR spectrum as depicted in FIG. 9; a DSC calorimetrogram with a broad endothermic peak at about 153° C. due to desolvation and a sharp exothermic melting peak at about 214° C.; a DSC calorimetrogram as depicted in FIG. 10; a TGA thermogram as depicted in FIG. 11, which shows a weight loss of about 0.3% up to 92° C. and a sharp weight loss of about 7.7% between 120° C. and 160° C. due to desolvation; and a combination thereof.

The above crystalline dolasetron trifluoroacetate Form 3 may be further characterized as an isopropanolate solvate in non-stoichiometric amounts. Preferably, the amount of propanol is about 6.3% by weight, as measured by GC or TGA.

The above crystalline dolasetron trifluoroacetate Form 3 has less than about 10% by weight, more preferably less than about 5% by weight, and most preferably less than about 1% by weight of crystalline dolasetron trifluoroacetate Forms 1, 2, 4 or mixtures thereof. The content of crystalline dolasetron trifluoracetate Form 1 in crystalline Form 3 is measured by PXRD using any one of the peaks at about 16.40, and 16.6±0.2 degrees two-theta; the content of crystalline Form 2 in crystalline Form 3 is measured by PXRD using the peak at about 12.0±0.2 degrees two-theta; and the content of crystalline Form 4 in crystalline Form 3 is measured by PXRD using the peak at about 9.0±0.2 degrees two-theta.

The above crystalline dolasetron trifluoroacetate Form 3 can be prepared by a process comprising crystallizing dolasetron trifluoroacetate from isopropanol.

Preferably, the crystallization comprises providing a solution of dolasetron trifluoroacetate in isopropanol, and precipitating the crystalline dolasetron trifluoroacetate to obtain a suspension.

Preferably, the solution of dolasetron trifluoroacetate in isopropanol is provided by combining dolasetron trifluoroacetae and isopropylalcohol to obtain a mixture, and heating the mixture. Preferably, heating is carried out to about reflux. Preferably, reflux is at a temperature of about 70° C. to about 90° C., more preferably at about 80° C. to about 85° C.

Preferably, precipitation is carried out by a process comprising cooling the solution to a temperature of about 20° C. to about −10° C., more preferably, to about 10° C. to about 0° C., most preferably, at about 8° C. to about 2° C. to obtain the suspension comprising said crystalline dolasetron trifluoroacetate.

Preferably, the suspension is further maintained at the same temperature for about 1 hour to about 24 hours, more preferably, for about 6 hours to about 20 hours, most preferably, for about 8 hours to about 16 hours, to increase the yield of the precipitated form.

The process for preparing the crystalline dolasetron trifluoroacetate Form 3 may further comprise recovering the crystalline dolasetron trifluoroacetate from the suspension. The recovery may be carried out for example, by filtering the suspension and drying the obtained material. Preferably, drying is conducted at a temperature of about 40° C. to about 120° C., more preferably at about 50° C. to about 100° C., most preferably at about 75° C. to about 85° C. Preferably, drying is carried out for about 1 hour to about 24 hours, more preferably for about 6 hours to about 20 hours, most preferably for about 8 hours to about 16 hours.

The present invention encompasses crystalline dolasetron trifluoroacetate, designated Form 4, characterized by data selected from the group consisting of: a powder XRD pattern with peaks at about 9.0, 18.1, 18.5, 19.4, and 20.4±0.2 degrees 2-theta, a powder XRD pattern as depicted in FIG. 12; and a combination thereof.

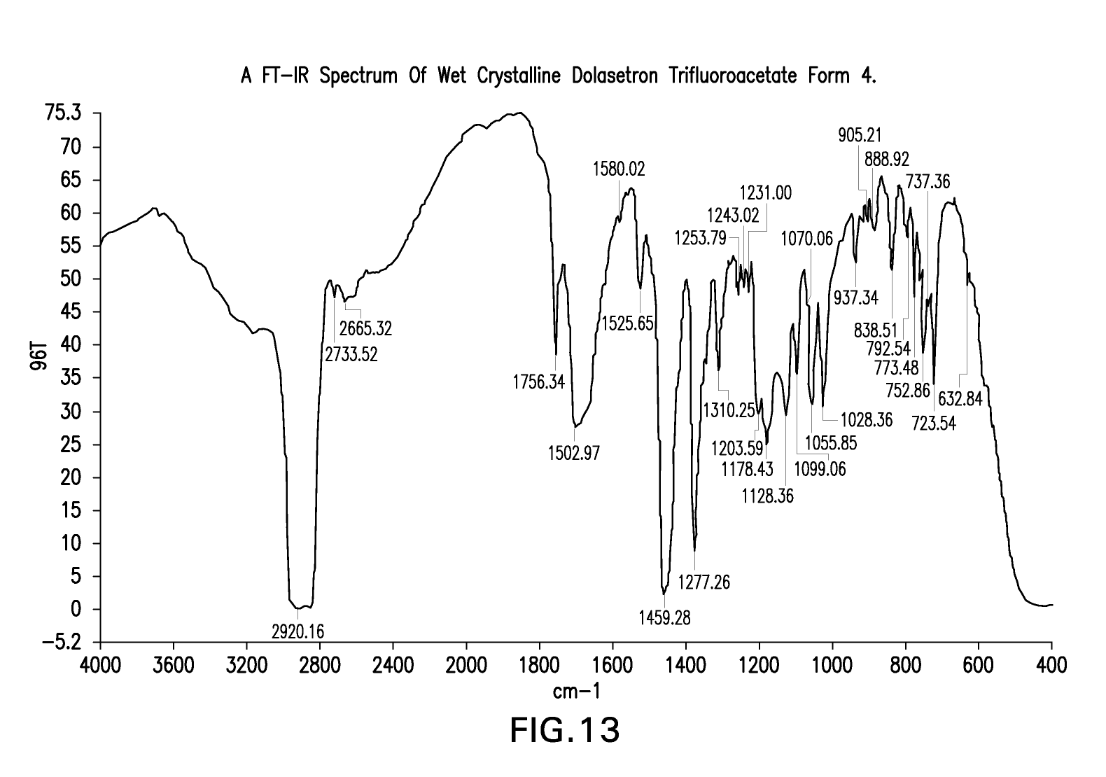

The above crystalline Form 4 of dolasetron trifluoroacetate can be further characterized by a powder XRD pattern with peaks at about 11.9, 12.5, 19.4, 21.8, 22.2, and 27.2±0.2; a FT-IR spectrum with peaks at about 2723, 1757, 1703, 1526, 1310, 1204, 1178, 1128, 839 and 723±2 cm⁻¹; a FT-IR spectrum as depicted in FIG. 13; and a combination thereof.

The above crystalline dolasetron trifluoroacetate Form 4 has less than about 10% by weight, more preferably less than about 5% by weight, and most preferably less than about 1% by weight of crystalline dolasetron trifluoroacetate Forms 1, 2, 3 or mixtures thereof. The content of crystalline dolasteron trifluoroacetate Form 1 in crystalline Form 4 is measured by PXRD using the peak at about 19.1±0.2 degrees two-theta; the content of crystalline Form 2 in crystalline Form 4 is measured by PXRD using the peak at about 16.9±0.2 degrees two-theta; the content of crystalline Form 3 in crystalline Form 4 is measured by PXRD using the peak at about 5.0±0.2 degrees two-theta.

The above crystalline dolasetron trifluoroacetate Form 4 can be prepared by a process comprising crystallizing dolasetron trifluoroacetate from ethanol.

Preferably, the crystallization comprises providing a solution of dolasetron trifluoroacetate in ethanol, and precipitating the crystalline dolasetron trifluoroacetate to obtain a suspension.

Preferably, the solution of dolasetron trifluoroacetate in ethanol is provided by combining dolasetron trifluoroacetate and ethanol to obtain a mixture and heating the mixture. Preferably, heating is carried out to about reflux. Preferably, reflux is at a temperature of about 70° C. to about 90° C., more preferably at about 75° C. to about 82° C.

Preferably, precipitation is carried out by a process comprising cooling the solution to a temperature of about 20° C. to about −10° C., more preferably, to about 10° C. to about 0° C., most preferably, to about 8° C. to about 2° C. to obtain the suspension comprising of the crystalline dolasetron trifluoroacetate Form 4.

Preferably, the suspension is further maintained at the same temperature for about 1 hour to about 24 hours, more preferably, for about 6 hours to about 20 hours, most preferably, for about 8 hours to about 16 hours to increase the yield of the precipitated Form.

The process for preparing the crystalline dolasetron trifluoroacetate Form 4 may further comprise recovering the crystalline dolasetron trifluoroacetate from the suspension. The recovery of crystalline dolasetron trifluoroacetate Form 4 may be carried out by a method that does not include drying, even when carried out at room temperature. Such methods include, but are not limited to, filtering the suspension.

In another embodiment, the present invention encompasses a process for preparing dolasetron mesylate from any one of the above crystalline Forms of dolasetron trifluoroacetate or mixtures thereof.

In yet another embodiment, the present invention provides a process for preparing dolasetron mesylate, by preparing any one of the above forms of dolasetron trifluoroacetate or mixtures thereof according to the process of the present invention, and converting them to dolasetron mesylate.

The conversion of dolasetron trifluoroacetate to dolasetron mesylate may be carried out, for example, by combining dolasetron trifluoroacetate with an inorganic base to obtain dolasetron base; and reacting dolasetron base with methanesulfonic acid to obtain dolasetron mesylate.

The preparation of dolasetron base is carried out in the presence of an organic solvent, preferably, C₁-C₅ esters or C₄-C₆ water-immiscible ketones. Preferably, the C₁-C₅ ester is iso-butyl acetate, n-butyl acetate, or ethyl acetate, more preferably, ethyl acetate. Preferably, the C₄-C₆ water-immiscible ketone is methyl ethyl ketone (2-butanone), 2-pentanone, 3-pentanone, methyl isobutyl ketone (4-methyl-2-pentanone) or methyl tert-butyl ketone (3,3-dimethyl-2-butanone), more preferably, methyl isobutyl ketone. Preferably, the inorganic base is an alkali metal carbonate, more preferably, sodium carbonate, most preferably, an aqueous solution of sodium carbonate.

Preferably, the reaction of dolasetron trifluoroacetate and the inorganic base is carried out while heating. Preferably, heating is to a temperature of about 20° C. to about 75° C., more preferably to about 30° C. to about 70° C., most preferably, to about 55° C. to about 65° C. Preferably, heating provides a solution comprising an aqueous phase and an organic phase.

Further, the phases are separated, and the organic phase is concentrated providing a suspension. The suspension is combined with a water-immiscible solvent, usually used for azeotropic distillation. Preferably, the water-immiscible solvent is chosen from a group consisting of xylene, tetrahydronaphtalene, dibutyl ether and toluene, more preferably, the water-immiscible solvent is toluene. A second suspension is obtained, comprising the water-immiscible solvent, ethylacetate, water and dolasetron base. The ethyl acetate and water are removed from the second suspension, to obtain a third suspension comprising said dolasetron base. Preferably, removing water and ethylacetate from the second suspension comprises evaporating the solvents from the second suspension and resuspending the resulting material in toluene. The process of evaporating and resuspension as above may be repeated one to five times to remove water and ethylacetate from the second suspension to obtain a third suspension in toluene.

The third suspension is then cooled. Preferably, cooling is carried out to a temperature of about 40° C. to about 0° C., more preferably to about 30° C. to about 10° C., most preferably to about 25° C. to about 15° C.

The process may further comprise recovering the obtained dolasetron base from the third suspension. The recovery may be carried out by filtration of the cooled suspension, and drying the filtered product. Preferably, drying is carried out at a temperature of about 40° C. to about 120° C., more preferably to about 50° C. to about 80° C., most preferably to about 55° C. to about 65° C. Preferably, drying is carried out for a period of about 0.5 hour to about 16 hours, more preferably for about 1 hour to about 10 hours, most preferably for about 2 hours to about 8 hours.

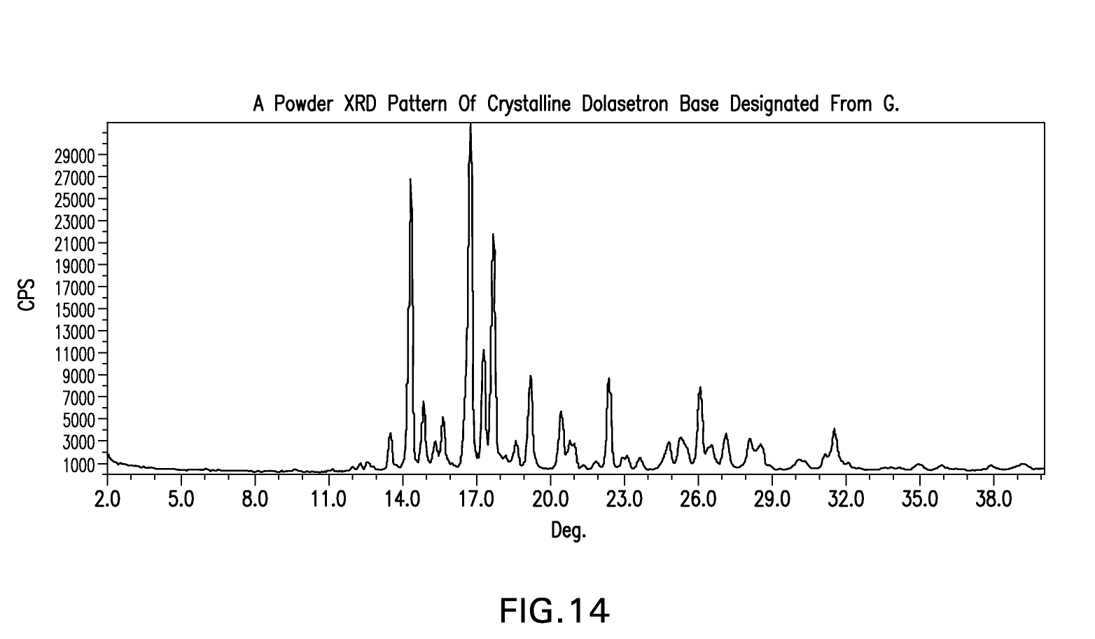

Preferably, the obtained dolasetron base is crystalline dolasetron base, more preferably, crystalline dolasetron base designated Form G, which is characterized by data selected from the group consisting of: a powder XRD pattern with peaks at about 14.3, 14.9, 16.7, 17.3, and 17.7±0.2 degrees 2-theta, a powder XRD pattern as depicted in FIG. 14; and combinations thereof.

The reaction of dolasetron base and methanesulfonic acid providing dolasetron mesylate comprises, combining the obtained dolasetron base, methanesulfonic acid, and a solvent mixture comprising acetone and water. Preferably, methane sulfonic acid is added to a suspension of the dolasetron base in a mixture comprising acetone and water. Preferably, the ratio of acetone and water is about 99:1 to about 80:20, preferably about 95:5.

Preferably, the addition of methane sulfonic acid transforms the suspension into a solution, in which a precipitate is formed after a few minutes.

The suspension is cooled to increase the yield of the precipitated dolasetron mesylate (monohydrate). Preferably, the suspension is cooled to a temperature of about 0° C. to about 10° C., more preferably to about 2° C. to about 8° C. Preferably, cooling is conducted for a period of about an hour to about 24 hours, more preferably for about 2 to about 8 hours, most preferably for about 4 hours.

The process may further comprise recovering the dolasetron mesylate (monohydrate). The recovery may be carried out for example by filtration of the cooled suspension, washing the filtered product, and drying it.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to limit its scope in any way.

### EXAMPLES

### Differential Scanning Calorimetry (DSC)

DSC 822^{e}/700, Mettler Toledo, Sample weight: 3-5 mg.
Heating rate: 10° C./min., Number of holes of the crucible: 3
In N₂ stream: flow rate=40 ml/min
Scan range: 30-250° C., 10° C./minutes heating rate.

### Thermal Gravimetric Analysis (TGA)

TGA/SDTA 851^{e}, Mettler Toledo, Sample weight 7-15 mg.
Heating rate: 10° C./min., In N₂ stream: flow rate=50 ml/min
Scan range: 25-250° C.

### NMR

Instrument: Bruker DRX 500 (500.13 MHz ¹H frequency, 125.78 MHz ¹³C frequency)
Solvent: DMSO-d6 reference for ppm scale

### Temperature: 300 K

### FT-IR Spectroscopy

Perkin-Elmer Spectrum One Spectrometer, at 4 cm⁻¹ resolution with 16 scans, in the range of 4000-400 cm⁻¹. Samples were analysed in Nujol mull. The spectra were recorded using an empty cell as a background.

### PXRD

ARL X-ray powder diffractometer model X'TRA-030, Peltier detector, round standard aluminum sample holder with round zero background quartz plate was used. Scanning parameters: Range: 2-40 deg. 2θ, continuous Scan, Rate: 3 deg./min. The accuracy of peak positions is defined as +/−0.2 degrees due to experimental differences like instrumentations, sample preparations etc.

### Example 1

### Preparation of Crystalline Dolasetron Trifluoroacetate Form 1

Indole-3-carboxylic acid (3.54 g, 1.1 equiv.) was added in portions to a solution of trifluoroacetic anhydride (4.0 ml, 1.4 equiv.) in a mixture toluene (70 ml) and trifluoroacetic acid (18 ml), at room temperature (20-25° C.), during 15 minutes. After 5-minutes of stirring, endo-5-hydroxy-8-azatricyclo[5.3.1.0^{3,8}]-undecan-10-one (3.62 g, 20 mmol), was added in one portion. The solution was stirred for 2 hours without external heating. The trifluoroacetic acid was removed by evaporation under reduced pressure, the crude dolasetron trifluoroacetate precipitated. The salt was filtered off, washed with toluene (10 ml), dried in vacuum at 80° C. for 4 hours. Polymorphism was determined by X-ray diffraction.

### Example 2

### Preparation of Crystalline Dolasetron Trifluoroacetate Form 2

Crystalline dolasetron trifluoroacetate, designated Form 4, characterized by data selected from the group consisting of: a powder XRD pattern with peaks at about 9.0, 11.9, 12.5, 18.1, 18.5, 19.4, 20.4, 21.8, 22.2, and 27.2±0.2 degrees 2-theta, a PXRD pattern as depicted in FIG. 11; a FT-IR spectrum with peaks at about 2723, 1757, 1703, 1526, 1310, 1204, 1178, 1128, 839 and 723 cm⁻¹; a FT-IR spectrum as depicted in FIG. 12, and a combination thereof was dried in vacuum at 80° C. overnight. Polymorphism was determined by X-ray diffraction.

### Example 3

### Preparation of Crystalline Dolasetron Trifluoroacetate Form 2

Dolasetron trifluoroacetate (8 g) was dissolved in ethanol (20 ml) under reflux. The solution was cooled to 2-8° C. and allowed to crystallize at this temperature overnight. The crystals were filtered off and dried in vacuum at 80° C. overnight. Polymorphism was determined by X-ray diffraction.

### Example 4

### Preparation of Crystalline Dolasetron Trifluoroacetate Form 3

Dolasetron trifluoroacetate (10 g) was dissolved in isopropanol (200 ml) under reflux. The solution was cooled to 2-8° C. and allowed to crystallize at this temperature overnight. The crystals were filtered off, dried in vacuum at 80° C. overnight. Polymorphism was determined by X-ray diffraction.

### Example 5

### Preparation of Crystalline Dolasetron Trifluoroacetate Form 4

Dolasetron trifluoroacetate (8 g) was dissolved in ethanol (20 ml) under reflux. The solution was cooled to 2-8° C. and allowed to crystallize at this temperature overnight. The crystals were filtered off. Polymorphism was determined by X-ray diffraction.

### Example 6

### Preparation of DLS Base from DLS-Trifluoroacetate

A mixture of dolasetron trifluoroacetate (5 g), ethyl acetate, (200 ml), and 10% of aqueous sodium carbonate (50 ml) was heated to 60° C., and stirred at this temperature until complete dissolution of solid material. After separation of phases the organic phase was washed with brine (50 ml). The ethyl acetate phase was evaporated to small volume (until obtaining a stirrable suspension) under atmospheric pressure. Toluene (100 ml) was added to the slurry and the mixture was evaporated to small volume. Again, toluene (100 ml) was added to the slurry and the mixture was evaporated to small volume. The suspension was cooled to room temperature, and the crystals were filtered off, dried in vacuum at 60° C. for 4 hours.

### Example 7

### Preparation of DLS-MsOH—H₂O

Methanesulfonic acid (2.85 ml, 1 equiv) was added to a stirred suspension of dolasetron base (14.24 g, 43.9 mmol) in a mixture of acetone-water 95:5 (100 ml). The solid dissolved immediately, after some minutes the salt precipitated in crystalline form. The mixture was put into a fridge, after 4 hours the salt was filtered off, washed with the same solvent mixture (2×15 ml), and dried overnight in an air-ventilated oven at 40° C.

What is claimed is:

1. Dolasetron trifluoroacetate.

2. The dolasetron trifluoroacetate of claim 1, wherein the dolasetron trifluoroacetate is isolated.

3. Dolasetron trifluoroacetate of claim 1, wherein the dolasetron trifluoroacetate is solid.

4. Dolasetron trifluoroacetate of claim 3, wherein the dolasetron trifluoroacetate is crystalline.

5. Dolasetron trifluoroacetate of claim 1, characterized by data selected from the group comprising a ¹H-NMR (500.13 MHz) spectrum in (CD₃)₂SO having chemical shifts at about δ 2.27-2.63 (m, 9H); 2.78 (s, 1H), 4.00 (m, 2H); 4.10 (s, 2H); 5.40 (s, 1H); 7.22 (m, 2H), 7.55 (m, 1H); 8.08 (m, 2H); 12.14 (s, 1H), a ¹³C-NMR (125.78 MHz) spectrum in (CD₃)₂SO having chemical shifts at about δ 25.37, 31.72, 37.54, 49.45, 58.96, 62.80, 105.84, 112.48, 120.02, 121.40, 122.52, 125.92, 132.45, 136.49, 158.86, 159.21, 162.93, 204.89; and a combination thereof.

6. Crystalline dolasetron trifluoroacetate characterized by data selected from the group consisting of: a powder XRD pattern with peaks at about 11.4, 17.2, and 18.5±0.2 degrees 2-theta, a powder XRD pattern as depicted in FIG. 7; and a combination thereof.

7. Crystalline dolasetron trifluoroacetate of claim 6, characterized by a powder XRD pattern with peaks at about 11.4, 17.2, and 18.5±0.2 degrees 2-theta.

8. Crystalline dolasetron trifluoroacetate of claim 6, characterized by a powder XRD pattern as depicted in FIG. 7.

9. Crystalline dolasetron trifluoroacetate of claim 6 further characterized by a powder XRD pattern with peaks at about 5.0, 9.3, 15.7, 16.9, 17.5, 19.4, and 20.3±0.2 degrees 2-theta.

10. Crystalline dolasetron trifluoroacetate of claim 6 further characterized by FT-IR peaks at about 3560, 1755, 1676, 1527, 1269, 1198, 1178, 1131, 1058 and 1026 cm⁻¹.

11. Crystalline dolasetron trifluoroacetate of claim 6 further characterized by a FT-IR spectrum as depicted in FIG. 8.

12. Crystalline dolasetron trifluoroacetate of claim 6 further characterized by a DSC calorimetrogram with an endothermic peak at about 153° C. and an exothermic peak at about 214° C.

13. Crystalline dolasetron trifluoroacetate of claim 6 further characterized by a DSC calorimetrogram as depicted in FIG. 9.

14. Crystalline dolasetron trifluoroacetate of claim 6 further characterized by a TGA thermogram as depicted in FIG. 10.

15. Crystalline dolasetron trifluoroacetate of claim 6 having less than about 10% by weight of crystalline dolasetron trifluoroacetate selected from the group consisting of: a powder XRD pattern with peaks at about 16.4, 16.6, 19.1 and, 21.6±0.2 degrees 2-theta, a powder XRD pattern as depicted in FIG. 1; and combination thereof, crystalline dolasetron trifluoroacetate characterized by data selected from the group consisting of: crystalline dolasetron trifluoroacetate characterized by data selected from a group consisting of: a powder XRD pattern with peaks at about 12.0, 15.3, 18.1, and 25.6±0.2 degrees 2-theta, a powder XRD pattern as depicted in FIG. 4; and combinations thereof, crystalline dolasetron trifluoroacetate characterized by data selected from the group consisting of: a powder XRD pattern with peaks at about 9.0, 18.1, 18.5, 19.4, and 20.4±0.2 degrees 2-theta, and a powder XRD pattern as depicted in FIG. 12; and combinations thereof, or mixtures thereof.

16. A process for preparing crystalline dolasetron trifluoroacetate characterized by data selected from the group consisting of: a powder XRD pattern with peaks at about 11.4, 17.2, and 18.5±0.2 degrees 2-theta, a powder XRD pattern as depicted in FIG. 7, and a combination thereof; comprising crystallizing dolasetron trifluoroacetate from isopropanol.

17. The process of claim 16 comprising providing a mixture of dolasetron trifluoroacetate and isopropanol, heating the mixture to obtain a solution and precipitating said crystalline dolasetron trifluoroacetate to obtain a suspension.

18. The process of claim 17, wherein the mixture of dolasetron trifluoroacetate and isopropanol is heated to a temperature of about 70° C. to about 90° C.

19. The process of claim 17 wherein precipitating comprises cooling the solution to a temperature of about 20° C. to about −10° C.

20. The process of claim 17 further comprising recovering the crystalline dolasetron trifluoroacetate from the suspension.

21. Dolasetron trifluoroacetate selected from the group consisting of: crystalline dolasetron trifluoroacetate characterized by a powder XRD pattern with peaks at about 16.4, 16.6, 19.1, and 21.6±0.2 degrees 2-theta, a powder XRD pattern as depicted in FIG. 1, and a combination thereof; and a crystalline dolasetron trifluoroacetate characterized by a powder XRD pattern with peaks at about 12.0, 15.3, 18.1, and 25.6±0.2 degrees 2-theta; a powder XRD pattern as depicted in FIG. 4, and a combination thereof.

22. Dolasetron trifluoroacetate of claim 21 characterized by a powder XRD pattern with peaks at about 16.4, 16.6, 19.1, and 21.6±0.2 degrees 2-theta.

23. Dolasetron trifluoroacetate of claim 21 characterized by a powder XRD pattern as depicted in FIG. 1.

24. Dolasetron trifluoroacetate of claim 21 characterized by a powder XRD pattern with peaks at about 12.0, 15.3, 18.1, and 25.6±0.2 degrees 2-theta.

25. Dolasetron trifluoroacetate of claim 21 characterized by a powder XRD pattern as depicted in FIG. 4.

26. A method of preparing dolasetron mesylate comprising converting dolasetron trifluoroacetate of any of claims 1 to 3 or crystalline dolasetron trifluoroacetate of any of claims 6 or 21 to dolasetron mesylate.

27. The process of claim 26, comprising combining the crystalline dolasetron trifluoroacetate with an inorganic base to obtain dolasetron base; and reacting dolasetron base with methanesulfonic acid to obtain dolasetron mesylate.

28. A process for preparing dolasetron mesylate comprising preparing crystalline dolasetron trifluoroacetate according to the processes of claim 16 and converting the obtained crystalline dolasetron trifluoroacetate to dolasetron mesylate.
